(19) United States
(12) Patent Application Publication
(10) Pub. No.: US 2013/0237451 A1
(43) Pub. Date: Sep. 12, 2013

(54) COMPOSITIONS AND METHODS FOR TREATMENT OF PROTEIN MISFOLDING DISEASES

(71) Applicant: WHITEHEAD INSTITUTE FOR BIOMEDICAL RESEARCH, Cambridge, MA (US)

(72) Inventors: Susan L. Lindquist, Cambridge, MA (US); Tiago Outeiro, Lisbon (PT)

(73) Assignee: Whitehead Institute for Biomedical Research, Cambridge, MA (US)

(21) Appl. No.: 13/685,384

(22) Filed: Nov. 26, 2012

Related U.S. Application Data
(63) Continuation of application No. 10/599,513, filed on Apr. 21, 2008, now abandoned.
(60) Provisional application No. 60/559,309, filed on Apr. 2, 2004.

(51) Int. Cl. C12Q 1/02

(52) U.S. Cl. CPC C12Q 1/025
USPC 506/10

(57) ABSTRACT

Disclosed are compounds and conditions that either suppress or enhance toxicity in yeast cells expressing alpha synuclein or huntingtin. These compounds and conditions can be used in the development of compositions that suppress toxicity, fibril formation, and/or diseases mediated at least in part by alpha synuclein or huntingtin.

### CROSS REFERENCE TO RELATED APPLICATIONS

This application claims priority from U.S. Provisional Application No. 60/559,309, filed Apr. 2, 2004. The entire content of the prior application is incorporated herein by reference in its entirety.

### STATEMENT AS TO FEDERALLY SPONSORED RESEARCH

This invention was made with Government support under grant number NS044829-01 awarded by the National Institutes of Health/National Institute for Neurological Disorders and Stroke. The Government may have certain rights in the invention.

### TECHNICAL FIELD

This invention relates to compositions and methods for treatment of protein misfolding diseases.

### BACKGROUND

Deposition of insoluble fibril proteins in tissues is a characteristic of diseases associated with protein misfolding. Exemplary protein misfolding diseases include neurodegenerative diseases (e.g., Parkinson's disease, Alzheimer's disease, Huntington's disease, and prion diseases) as well as diseases such as type 2 diabetes. Certain proteins such have been characterized as undergoing misfolding events in specific diseases (e.g., alpha synuclein in Parkinson's disease and huntingtin in Huntington's disease).

Parkinson's disease has a prevalence of about 2% after age 65, and, thus, is one of the most common neurodegenerative human disorders. Its pathological hallmarks are: (a) the presence of Lewy bodies (LBs) (Spillantini M G, et al., 1997. Nature 388:839-40), round cytoplasmic inclusions ˜5-25 μm in diameter, mainly reactive for alpha-synuclein but also for ubiquitin and other proteins; and (b) massive loss of dopaminergic neurons in the pars compacta of the substantia nigra (Fearnley J M, et al., 1991. Brain 114:2283-2301).

### SUMMARY

The invention is based, at least in part, on the discovery that certain compounds can suppress toxicity in yeast expressing alpha synuclein (aS) or huntingtin (htt). The invention is also based, at least in part, on the identification of conditions that can enhance toxicity in yeast expressing aS or htt. The identification of such compounds and conditions allows for the development of compositions that can suppress toxicity, fibril formation, and/or diseases mediated at least in part by aS or htt.

In one aspect, the invention features a method of inhibiting aS mediated toxicity by contacting a cell expressing aS with a composition containing an amount of a compound effective to inhibit aS mediated toxicity in the cell, wherein the compound is selected from the group consisting of nordihydroguaiaretic acid, ibuprofen, D,L-a-hydroxy-butyric acid, m-cresol, hexachlorophene, ruthenium red, sodium metasilicate, sodium metavanadate, sodium cyanide, and tetracycline.

The invention also features a method of inhibiting aS mediated toxicity by contacting a cell expressing aS with a composition containing an amount of a compound effective to inhibit aS mediated toxicity in the cell, wherein the compound is selected from the group consisting of a fungicide, lipoxygenase inhibitor, prostaglandin synthetase inhibitor, membrane detergent, electron transporter, mitochondrial Ca++ porter, toxic anion, and antibiotic.

The invention also features a method of inhibiting aS mediated fibril formation by contacting a cell expressing aS with a composition containing an amount of a compound effective to inhibit aS mediated fibril formation in the cell, wherein the compound is selected from the group consisting of nordihydroguaiaretic acid, ibuprofen, D,L-a-hydroxy-butyric acid, m-cresol, hexachlorophene, ruthenium red, sodium metasilicate, sodium metavanadate, sodium cyanide, and tetracycline.

The invention also features a method of inhibiting aS mediated fibril formation by contacting a cell expressing aS with a composition containing an amount of a compound effective to inhibit aS mediated fibril formation in the cell, wherein the compound is selected from the group consisting of a fungicide, lipoxygenase inhibitor, prostaglandin synthetase inhibitor, membrane detergent, electron transporter, mitochondrial Ca++ porter, toxic anion, and antibiotic.

In another aspect, the invention features a method of treating or preventing Parkinson's disease by administering to an individual in need thereof a pharmaceutical composition containing a therapeutically effective amount of a compound selected from the group consisting of nordihydroguaiaretic acid, ibuprofen, D,L-a-hydroxy-butyric acid, m-cresol, hexachlorophene, ruthenium red, sodium metasilicate, sodium metavanadate, sodium cyanide, and tetracycline.

The invention also features a method of treating or preventing Parkinson's disease by administering to an individual in need thereof a pharmaceutical composition containing a therapeutically effective amount of a compound selected from the group consisting of a fungicide, lipoxygenase inhibitor, prostaglandin synthetase inhibitor, membrane detergent, electron transporter, mitochondrial Ca++ porter, toxic anion, and antibiotic.

In another aspect, the invention features a method of inhibiting htt mediated toxicity by contacting a cell expressing htt with a composition containing an amount of a compound effective to inhibit htt mediated toxicity in the cell, wherein the compound is selected from the group consisting of a clioquinol (e.g., 8-Hydroxyquinoline, 5,7-Dichloro-8-hydroxy-quinaldine, and 8-Hydroxy-5-nitroquinoline), histidine-containing dipeptide, nordihydroguaiaretic acid, m-cresol, and guanidine hydrochloride.

The invention also features a method of inhibiting htt mediated toxicity by contacting a cell expressing htt with a composition containing an amount of a compound effective to inhibit htt mediated toxicity in the cell, wherein the compound is selected from the group consisting of a chelator, fungicide, lipoxygenase inhibitor, membrane detergent, and chaotropic agent.

The invention also features a method of inhibiting htt mediated fibril formation by contacting a cell expressing htt with a composition containing an amount of a compound effective to inhibit htt mediated fibril formation in the cell, wherein the compound is selected from the group consisting of a clioquinol (e.g., 8-Hydroxyquinoline, 5,7-Dichloro-8-hydroxy-quinaldine, and 8-Hydroxy-5-nitroquinoline), histidine-containing dipeptide, nordihydroguaiaretic acid, m-Cresol, and guanidine hydrochloride.

The invention also features a method of inhibiting htt mediated fibril formation by contacting a cell expressing htt with a composition containing an amount of a compound effective to inhibit htt mediated fibril formation in the cell, wherein the compound is selected from the group consisting of a chelator (e.g., a copper and/or zinc chelator), fungicide, lipoxygenase inhibitor, membrane detergent, and chaotropic agent.

In another aspect, the invention features a method of treating or preventing Huntington's disease by administering to an individual in need thereof a pharmaceutical composition containing a therapeutically effective amount of a compound selected from the group consisting of a clioquinol (e.g., 8-Hydroxyquinoline, 5,7-Dichloro-8-hydroxy-quinaldine, and 8-Hydroxy-5-nitroquinoline), histidine-containing dipeptide, nordihydroguaiaretic acid, m-Cresol, and guanidine hydrochloride.

The invention also features a method of treating or preventing Huntington's disease by administering to an individual in need thereof a pharmaceutical composition containing a therapeutically effective amount of a compound selected from the group consisting of a chelator, fungicide, lipoxygenase inhibitor, membrane detergent, and chaotropic agent.

In another aspect, the invention features a method of identifying a compound that inhibits aS mediated toxicity, the method including: (1) providing a yeast cell expressing an amount of aS that reduces viability of the cell; (2) contacting the cell with candidate agent selected from the group consisting of a fungicide, lipoxygenase inhibitor, prostaglandin synthetase inhibitor, membrane detergent, electron transporter, mitochondrial Ca++ porter, toxic anion, and antibiotic; and (3) determining whether the candidate agent enhances viability of the cell, to thereby identify a compound that inhibits aS mediated toxicity.

The invention also features a method of identifying a compound that inhibits htt mediated toxicity, the method including: (1) providing a yeast cell expressing an amount of htt that reduces viability of the cell; (2) contacting the cell with a candidate agent selected from the group consisting of a chelator, fungicide, lipoxygenase inhibitor, membrane detergent, and chaotropic agent; and (3) determining whether the candidate agent enhances viability of the cell, to thereby identify a compound that inhibits htt mediated toxicity.

The invention also features a method of identifying a compound that inhibits htt mediated toxicity, the method including: (1) providing a yeast cell expressing an amount of htt that reduces viability of the cell; (2) contacting the cell with a clioquinol; and (3) determining whether the clioquinol enhances viability of the cell, to thereby identify a compound that inhibits htt mediated toxicity.

The invention also features a method of identifying a compound that inhibits aS mediated toxicity, the method including: (1) identifying a candidate agent that stimulates the expression or activity of a protein encoded by a gene selected from the group consisting of CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, and SMARCA1; (2) contacting a cell expressing aS with the candidate agent; and (3) determining whether the candidate agent enhances viability of the cell, to thereby identify a compound that inhibits aS mediated toxicity.

The invention also features a method of identifying a compound that inhibits aS mediated toxicity, the method including: (1) providing a cell expressing aS and not expressing a wild type gene selected from the group consisting of CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, and SMARCA1, such that the cell has reduced viability as compared to a cell not expressing aS and expressing the wild type gene; (2) contacting the cell with a candidate agent; and (3) determining whether the candidate agent enhances viability of the cell, to thereby identify a compound that inhibits aS mediated toxicity.

The invention also features a method of identifying a compound that inhibits aS mediated toxicity, the method including: (1) identifying a candidate agent that modulates osmotic sensitivity or the activity of detergents, oxidants, or drugs affecting transport; (2) contacting a yeast cell expressing aS with the candidate agent; and (3) determining whether the candidate agent enhances viability of the cell, to thereby identify a compound that inhibits aS mediated toxicity.

In another aspect, the invention features a pharmaceutical composition containing a therapeutically effective amount of a compound described in the above methods.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts human genes having similarity (BLAST e-value less than 1e-50) to yeast genes identified in an alpha synuclein screen.

FIG. 2 depicts human genes having similarity (BLAST e-value less than 1e-30) to yeast genes identified in an alpha synuclein screen.

### DETAILED DESCRIPTION

Protein misfolding and/or protein fibril formation and/or protein aggregation may contribute to numerous neurodegenerative diseases (e.g., Parkinson's disease, Parkinson's Disease with accompanying dementia, dementia with Lewy bodies, Alzheimer's Disease, Alzheimer's Disease with Parkinsonism, multiple system atrophy (MSA), Huntington's Disease, spinocerebellar ataxia (SCA), and prion diseases) as well as non-neuronal diseases (e.g., type 2 diabetes). Yeast cells that ectopically expressing such a misfolded protein, which can be a wildtype or a mutant protein, are useful for identifying candidate drugs which inhibit misfolding and/or abnormal processing of proteins and, thus, are useful for therapy (prevention, treatment, including inhibition of progression and reversal) of protein misfolding diseases.

Parkinson's disease (PD) is one example of a protein misfolding disease. Studies of the genetic basis of PD identified two missense mutations in the alpha-synuclein gene (Kruger R, et al., 1998. Nat. Genet. 18, 106-108; Polymeropoulos M H, et al., 1997. Science 276, 2045-2047). One of these mutations is a substitution of an alanine for a threonine at position 53 (A53T), the other is an alanine for a proline at position 30 (A30P). Alpha synuclein was the first “PD gene” to be discovered, and it may also be involved in the pathogenesis of other neurodegenerative diseases, such as Alzheimer's disease and multiple system atrophy.

### Yeast Cells

Yeast (e.g., Saccharomyces cerevisiae) has become an extraordinarily powerful system for studying complex biological problems. There are numerous advantaged to using yeast as a model system. These include: 1) switching readily between haploid and diploid genetics; 2) the ease of site directed mutagenesis; 3) the availability of many expression vectors; 4) methods for genetic and chemical screens that can be performed at a fraction of the price in time and materials required in other systems; 5) a chaperone machinery, particularly relevant for problems involving protein folding, that is extensively characterized; and 6) special strains with greatly enhanced drug sensitivities. Finally, because the yeast genome was the first eukaryotic genome to be sequenced it is currently the single best-characterized eukaryotic cell. Yeast can be used as a model system or living test tubes for studying protein misfolding (see, e.g., Outeiro et al. (2003) Science 302:1772).

A wide variety of yeast strains may be used in the methods described herein. Strains that can be used include, but are not limited to, Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp. and Geotrichum fermentans. Although much of the discussion herein relates to Saccharomyces cerevisiae which ectopically expresses an abnormally processed protein, this is merely for illustrative purposes. Other yeast strains can be substituted for S. cerevisiae.

Certain mutations of yeast strains enhance uptake of candidate agents by yeast cells, decrease metabolism of a candidate agent after it enters a yeast cell, or decrease a candidate agent's being pumped out of a yeast cell. For example, a yeast strain bearing mutations in the ERG6 gene, the PDR1 gene, and/or the PDR3 gene, which affect membrane efflux pumps and increasing permeability for drugs are contemplated of use.

### Alpha Synuclein

In certain aspects, the methods relate to the use of an alpha synuclein protein. In some embodiments, a full-length wild type human alpha synuclein protein may be used. The term “full-length” refers to an alpha synuclein protein that contains at least all the amino acids encoded by the wild type human alpha synuclein cDNA. In other embodiments, different lengths of the alpha synuclein protein may be used. For example, only functionally active domains of the protein may be used. Thus, a protein fragment of almost any length may be employed.

In certain embodiments, mutants or variants of the aS protein can be used. Such variants may include biologically-active fragments of the aS protein. These include proteins with aS activity that have amino acid substitutions. In certain embodiments, aS mutants are ectopically expressed in yeast include the A53T mutant (containing a substitution of an alanine for a threonine at position 53) and the A30P mutant (containing a substitution of an alanine for a proline at position 30).

In certain embodiments, fusion proteins including at least a portion of the aS protein or a mutant aS may be used. For example, a portion of the aS protein may be fused with a second domain. The second domain of the fusion proteins can be selected from the group consisting of: an immunoglobulin element, a dimerizing domain, a targeting domain, a stabilizing domain, and a purification domain. Alternatively, a portion of aS protein can be fused with a heterologous molecule such as a detection protein. Exemplary detection proteins include: (1) a fluorescent protein such as green fluorescent protein (GFP), cyan fluorescent protein (CFP) or yellow fluorescent protein (YFP); (2) an enzyme such as β-galactosidase or alkaline phosphatase (AP); and (3) an epitope such as glutathione-S-transferase (GST) or hemagluttin (HA). To illustrate, an alpha synuclein protein can be fused to GFP at the N- or C-terminus or other parts of the aS protein. These fusion proteins provide methods for rapid and easy detection and identification of the aS protein in the recombinant host cell.

### Nucleic Acid Vectors for Expression in Yeast

A gene encoding a component of an assay system (e.g., alpha synuclein or huntingtin) may be transfected into a yeast cell using nucleic acid vectors that include, but are not limited to, plasmids, linear nucleic acid molecules, artificial chromosomes, and episomal vectors. Yeast plasmids are preferred and three well known systems used for recombinant plasmid expression and replication in yeast cells include integrative plasmids, low-copy-number ARS-CEN plasmids, and high-copy-number 2μ plasmids. See Sikorski, “Extrachromsomoal cloning vectors of Saccharomyces cerevisiae,” in Plasmid, A Practical Approach, Ed. K. G. Hardy, IRL Press, 1993; and Yeast Cloning Vectors and Genes, Current Protocols in Molecular Biology, Section II, Unit 13.4, Eds., Ausubel et al., 1994.

An example of the integrative plasmids is YIp, which is maintained at one copy per haploid genome, and is inherited in Mendelian fashion. Such a plasmid, containing a gene of interest, a bacterial origin of replication and a selectable gene (typically an antibiotic-resistance marker), is produced in bacteria. The purified vector is linearized within the selectable gene and used to transform competent yeast cells.

An example of the low-copy-number ARS-CEN plasmids is YCp, which contains the autonomous replicating sequence (ARS1) and a centromeric sequence (CEN4). These plasmids are usually present at 1-2 copies per cell. Removal of the CEN sequence yields a YRp plasmid, which is typically present in 100-200 copies per cell. However, this plasmid is both mitotically and meiotically unstable.

An example of the high-copy-number 2μ plasmids is YEp, which contains a sequence approximately 1 kb in length (named the 2μ sequence). The 2μ sequence acts as a yeast replicon giving rise to higher plasmid copy number. However, these plasmids are unstable and require selection for maintenance. Copy number is increased by having on the plasmid a selection gene operatively linked to a crippled promoter.

A wide variety of plasmids can be used in the present methods. In one embodiment, the plasmid is an integrative plasmid (e.g., pRS303, pRS304, pRS305 or pRS306 or other integrative plasmids). In further embodiments, the plasmid is an episomal plasmid (e.g., p426GPD, p416GPD, p426TEF, p423GPD, p425GPD, p424GPD or p426GAL).

Regardless of the type of plasmid used, yeast cells are typically transformed by chemical methods (e.g., as described by Rose et al., 1990, Methods in Yeast Genetics, Cold Spring Harbor Laboratory Press, Cold Spring Harbor, N.Y.). The cells are typically treated with lithium acetate to achieve transformation efficiencies of approximately 10⁴ colony-forming units (transformed cells)/μg of DNA. Yeast perform homologous recombination such that the cut, selectable marker recombines with the mutated (usually a point mutation or a small deletion) host gene to restore function. Transformed cells are then isolated on selective media.

The yeast vectors (plasmids) used in the disclosed methods typically comprise a yeast origin of replication, an antibiotic resistance gene, a bacterial origin of replication (for propagation in bacterial cells), multiple cloning sites, and a yeast nutritional gene for maintenance in yeast cells. The nutritional gene (or “auxotrophic marker”) is most often one of the following: 1) TRP1 (Phosphoribosylanthranilate isomerase); 2) URA3 (Orotidine-5′-phosphate decarboxylase); 3) LEU2 (3-Isopropylmalate dehydrogenase); 4) HIS3 (Imidazoleglycerolphosphate dehydratase or IGP dehydratase); or 5) LYS2 (α-aminoadipate-semialdehyde dehydrogenase).

Yeast vectors (plasmids) may also comprise promoter sequences. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively linked” and “operatively positioned” mean that a promoter is in a correct functional location and/or orientation in relation to a nucleic acid sequence to control transcriptional initiation and/or expression of that sequence.

A promoter may be one naturally associated with a nucleic acid sequence, as may be obtained by isolating the 5′ non-coding sequences located upstream of the coding segment and/or exon. Such a promoter can be referred to as “endogenous.” Alternatively, a promoter may be a recombinant or heterologous promoter, which refers to a promoter that is not normally associated with a nucleic acid sequence in its natural environment. Such promoters may include promoters of other genes and promoters not “naturally occurring.” The promoters employed may be either constitutive or inducible.

For example, various yeast-specific promoters (elements) may be employed to regulate the expression of a RNA in yeast cells. Examples of inducible yeast promoters include GAL1-10, GAL1, GALL, GALS, TET, VP16 and VP16-ER. Examples of repressible yeast promoters include Met25. Examples of constitutive yeast promoters include glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), and MRP7. Autonomously replicating expression vectors of yeast containing promoters inducible by glucocorticoid hormones have also been described (Picard et al., 1990), including the glucocorticoid responsive element (GRE). These and other examples are described in Mumber et al., 1995; Ronicke et al., 1997; Gao, 2000, all incorporated herein by reference. Yet other yeast vectors containing constitutive or inducible promoters such as alpha factor, alcohol oxidase, and PGH may be used. For reviews, see Ausubel et al. and Grant et al., 1987.

For example, 2μ vectors are present in high copy and permit high levels of expression, but they have the disadvantage of varying in number from cell to cell and instability. Integrating constructs are extremely stable but produce lower levels of expression. Constitutive promoters allow expression in normal media, but inducible promoters allow to control the levels and timing of expression. Controllable expression is of particular interest when dealing with potentially toxic proteins, to enhance transformation efficiencies and avoid the accumulation of mutations in the genome that alter aS function and toxicity.

### Screening Assays

Certain aspects of the present invention provide methods (assays) of screening for a candidate drug (agent or compound) and identifying a drug for treating a protein folding disease. A “candidate drug” or a “candidate agent” as used herein, is any substance with a potential to reduce, interfere with or block activities/functions of an abnormally processed protein (e.g., alpha-synuclein or huntingtin). Various types of candidate drugs may be screened, including nucleic acids, polypeptides, small molecule compounds, and peptidomimetics. In some cases, genetic agents can be screened by contacting the yeast cell with a nucleic acid construct encoding for a gene. For example, one may screen cDNA libraries expressing a variety of genes, to identify therapeutic genes for the diseases described herein. In other examples, one may contact the yeast cell with other proteins or polypeptides which may confer the therapeutic effect. For example, the identified drugs may prevent aS or htt mediated toxicity in a cell.

In certain embodiments, the screening methods use yeast cells that are engineered to express a protein (e.g., an aS protein or an htt protein).

For chemical screens, suitable mutations of yeast strains can be used that are designed to affect membrane efflux pumps and increase permeability for drugs. For example, a yeast strain bearing mutations in the ERG6 gene, the PDR1 gene, and/or the PDR3 gene is contemplated of use. For example, a yeast strain bearing mutations in membrane efflux pumps (erg6, pdr1 and pdr3) has been successfully used in many screens to identify growth regulators (Jensen-Pergakes K L, et al., 1998. Antimicrob Agents Chemother 42:1160-7).

In certain embodiments, candidate drugs can be screened from large libraries of synthetic or natural compounds. One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can immediately be used in the screens.

Potential drugs may include a small molecule. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In certain embodiments, such candidate drugs also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulphydryl or carboxyl group.

Other suitable candidate drugs may include antisense molecules, ribozymes, and antibodies (including single chain antibodies), each of which would be specific for the target molecule. For example, an antisense molecule that binds to a translational or transcriptional start site, or splice junctions, would be ideal candidate inhibitors.

In one embodiment, the invention contemplates screening assays using fluorescent resonance energy transfer (FRET). FRET occurs when a donor fluorophore is in close proximity (10-60 A) to an acceptor fluorophore, and when the emission wavelength of the first overlaps the excitation wavelength of the second (Kenworthy A K et al., 2001. Methods. 24:289-96). FRET should occur when cyan fluorescent protein (CFP) and yellow fluorescent protein (YFP) fusion proteins are actually part of the same complex.

For example, an alpha-synuclein protein is fused to CFP and to YFP respectively, and is integrated in the yeast genome under the regulation of a GAL1-10 promoter. Cells are grown in galactose to induce expression. Upon induction, cells produce the fusion proteins, which aggregate and bring the CFP and YFP close together. Because proteins in the aggregates are tightly packed, the distance between the CFP and YFP is less than the critical value of 100 A that is necessary for FRET to occur. In this case, the energy released by the emission of CFP will excite the YFP, which in turn will emit at its characteristic wavelength. The present inventors contemplate utilizing FRET based screening to identify candidate compounds including, drugs, genes or other factors that can disrupt the interaction of CFP and YFP by maintaining the proteins in a state that does not allow aggregation to occur.

In one embodiment, the invention contemplates screening assays using fluorescence activated cell sorting (FACS) analysis. FACS is a technique well known in the art, and provides the means of scanning individual cells for the presence of fluorescently labeled/tagged moiety. The method is unique in its ability to provide a rapid, reliable, quantitative, and multiparameter analysis on either living or fixed cells. For example, the misfolded aS protein can be suitably labeled, and provide a useful tool for the analysis and quantitation of protein aggregation and fibril and/or aggregate formation as a result of other genetic or growth conditions of individual yeast cells as described above.

In particular embodiments, methods of the present invention relate to determining aS associated toxicity. One of the strongest aspects of yeast is the possibility of performing high throughput screens that may identify genes, peptides and other compounds with the potential to ameliorate toxicity. A large number of compounds can be screened under a variety of growth conditions and in a variety of genetic backgrounds. The toxicity screen has the advantage of not only selecting for compounds that interact with aS, but also upstream or downstream targets that are not themselves cytotoxic and that are not yet identified.

For example, the Bioscreen-C system (Labsystem Corp, Helsinki, Finland) permits the growth of up to 200 cell cultures at the same time, under different conditions. Growth rates are monitored optically, recorded automatically, and stored as digital files for further manipulations. Growth will be monitored in the presence of genetic libraries, chemicals, drugs, etc. to identify those that give a selective growth advantage. Mutants and chemicals from a variety of sources will be tested.

### Screens for Suppressors of Toxicity

Several groups have shown that high levels of WT and A53T aS are toxic to mammalian cells (Ostrerova N, et al., 1999. J. Neurosci. 19:5782-5791; Zhou W, et al., 1999. Soc. Neurosci. 25:27.15). WT aS and A53T, but not A30P, are toxic in yeast. Cells expressing aS alone, or aS GFP, YFP and CFP fusions behave identically. Toxicity is dosage dependent. Cells that contain one integrated copy of an aS-GFP fusion gene under the regulation of a galactose-inducible promoter exhibit moderate growth defects whereas cells with two copies have extreme defects. Under conditions that repress expression (growth in glucose) there is no growth difference between strains carrying these constructs.

High levels of toxicity, with two aS integrated genes, provide the best mechanism for finding factors that reduce toxicity. Low levels of toxicity, with one copy, provide a more sensitive system for testing factors that modulate toxicity. Using the single copy, low expression strains we observed that cells expressing aS were far more susceptible certain stresses than control cells.

Yeast expressing a toxicity-inducing form and/or amount of a protein comprising aS or htt (or a biologically active fragment thereof) can be screened to identify compounds that rescue growth and inhibit toxicity mediated by aS or htt. An exemplary aS-expressing yeast cell for use in a growth rescue screen described herein is yeast expressing two copies of aS, as described in Outeiro et al. (2003) Science 302:1772. An exemplary htt-expressing strain for use in a growth rescue screen described herein is yeast expressing a fusion protein comprising a FLAG tag and an expanded htt polyQ (103) domain, as described in Meriin et al. (2002) J. Cell Biol. 158:591.

Screening of aS-expressing cells to identify compounds that inhibit aS mediated toxicity can be carried out with a candidate agent such as a fungicide, lipoxygenase inhibitor, prostaglandin synthetase inhibitor, membrane detergent, electron transporter, mitochondrial Ca++ porter, toxic anion, or antibiotic. Screening of htt-expressing cells to identify compounds that inhibit htt mediated toxicity can be carried out with a candidate agent such as a clioquinol, chelator, fungicide, lipoxygenase inhibitor, membrane detergent, or chaotropic agent. The loss of function of one or more of the following human genes is expected to enhance aS-mediated toxicity in cells: CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, or SMARCA1. Accordingly, screens can be carried out to identify a candidate agent that stimulates the expression or activity of a protein encoded by any of these genes. Such stimulatory candidate agents can then be used to evaluate their ability to enhance viability of a cell (e.g., a yeast cell) expressing aS.

As a result of the enhanced toxicity in aS-expressing cells that do not express at least one of the above genes, such genetically modified cells can be contacted with a candidate agent to determine whether the candidate agent enhances viability of the cell.

Screens can also be carried out to identify a candidate agent that modulates osmotic sensitivity or the activity of detergents, oxidants, or drugs affecting transport. Such candidate agents can then be used to evaluate their ability to enhance viability of a cell expressing aS.

### Formulation of Pharmaceutical Compositions and Methods of Treatment

The pharmaceutical compositions provided herein contain therapeutically effective amounts of one or more of the compounds provided herein that are useful in the prevention, treatment, or amelioration of one or more of the symptoms of diseases or disorders associated with α-synuclein or huntingtin fibril formation, or in which α-synuclein or huntingtin fibril formation is implicated, in a pharmaceutically acceptable carrier. Diseases or disorders associated with α-synuclein fibril formation include, but are not limited to, neurodegenerative diseases, including but not limited to Parkinson's Disease, Parkinson's Disease with accompanying dementia, Lewy body dementia, Alzheimer's disease with Parkinsonism, and multiple system atrophy. Pharmaceutical carriers suitable for administration of the compounds provided herein include any such carriers known to those skilled in the art to be suitable for the particular mode of administration.

In certain embodiments, the present invention provides methods of treating a subject (patient or individual) suffering from an aS associated disease (Parkinson's disease) and/or an htt associated disease (e.g., Huntington's disease). In other embodiments, the invention provides methods of preventing or reducing the onset of such diseases in a subject. For example, an individual who is at risk of developing Parkinson's disease or Huntington's disease (e.g., an individual whose family history includes Parkinson's disease or Huntington's disease) and/or has signs he/she will develop Parkinson's disease or Huntington's disease can be treated by the present methods. These methods comprise administering to the individual an effective amount of a compound described herein or a compound identified by a screening method as described herein. These methods are particularly aimed at therapeutic and prophylactic treatments of animals, and more particularly, humans.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with α-synuclein or huntingtin fibril formation or in which α-synuclein or huntingtin fibril formation is implicated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems well known to those of skill in the art and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with α-synuclein or huntingtin fibril formation or in which α-synuclein or huntingtin fibril formation is implicated, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In certain embodiments, one or more compositions can be administered with another type(s) of composition(s) for treating a protein misfolding disease. For example, the identified drug may be administered together with Levodopa (L-DOPA) for treating Parkinson's disease.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for modulating α-synuclein or huntingtin fibril formation, or for treatment, prevention or amelioration of one or more symptoms of α-synuclein or huntingtin mediated diseases or disorders, or diseases or disorders in which α-synuclein or huntingtin fibril formation, is implicated, within the packaging material, and a label that indicates that the compound or composition, or pharmaceutically acceptable derivative thereof, is used for modulating the α-synuclein or huntingtin fibril formation, or for treatment, prevention or amelioration of one or more symptoms of α-synuclein or huntingtin mediated diseases or disorders, or diseases or disorders in which α-synuclein or huntingtin fibril formation is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which α-synuclein or huntingtin fibril formation is implicated as a mediator or contributor to the symptoms or cause.

The following are examples of the practice of the invention. They are not to be construed as limiting the scope of the invention in any way.

### EXAMPLES

### Example 1

### Identification of Human Genes Similar to Yeast Genes Identified in Alpha Synuclein Screen

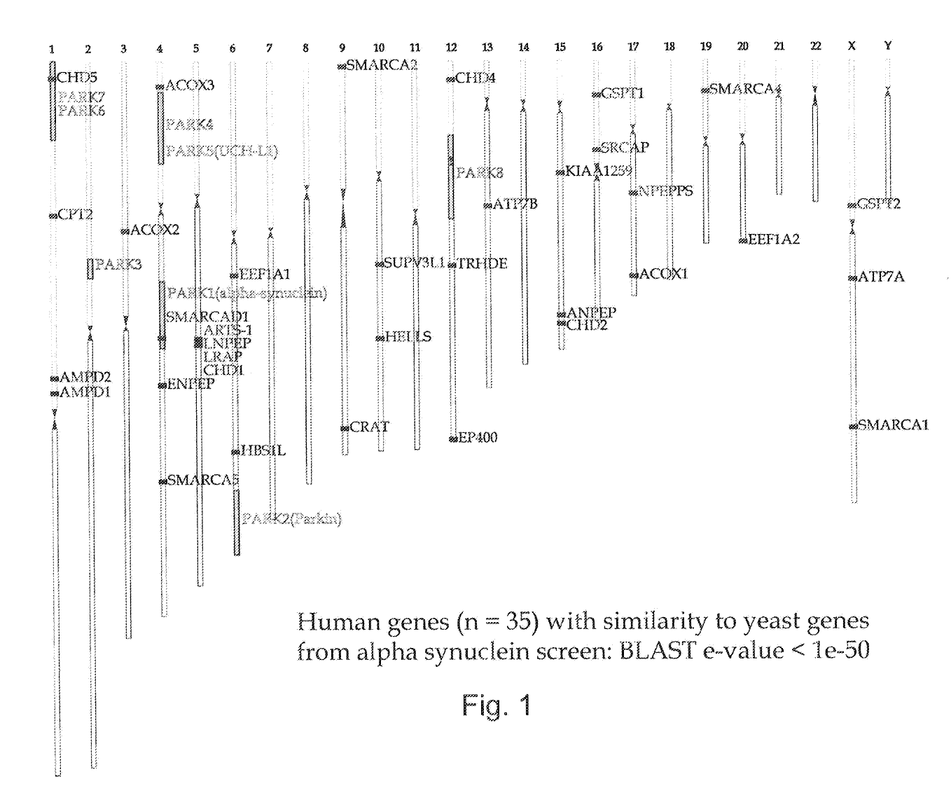

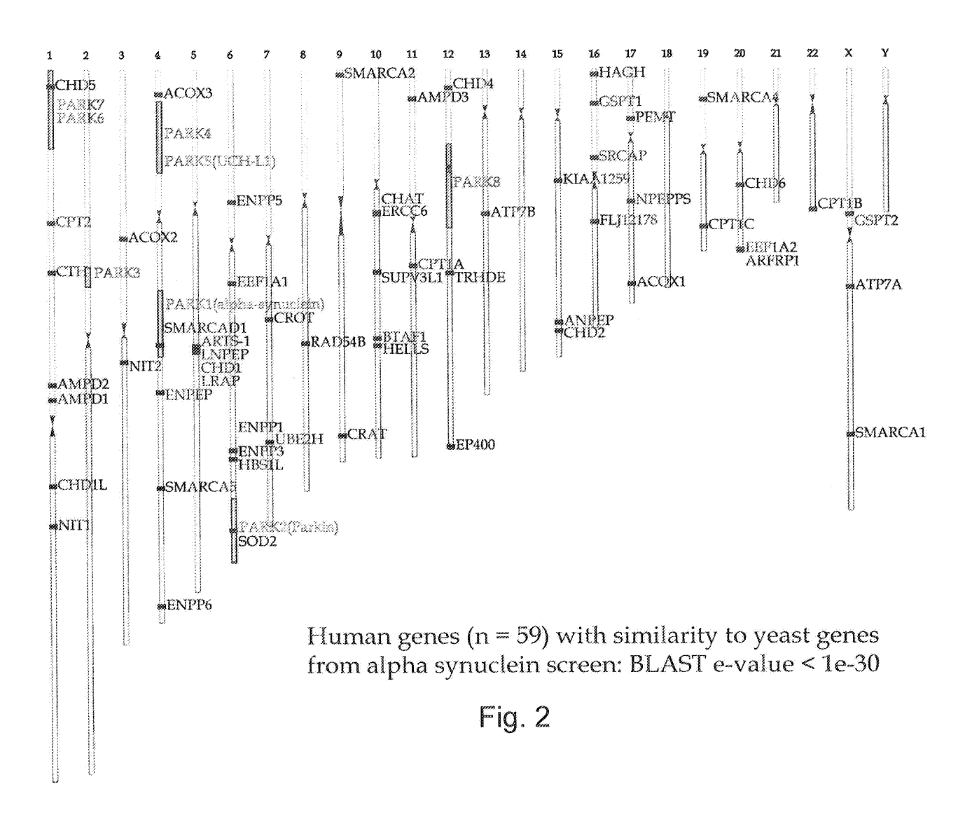

Alpha synuclein-expressing yeast were screened to identify genes (86 in total) that, upon loss of their function, enhanced alpha synuclein-mediated toxicity in yeast. Subsequently, the human RefSeq protein set was searched with the corresponding yeast proteins so as to identify human orthologs of the yeast genes identified by the alpha-synuclein screen. Genes encoding these human proteins were mapped in the human genome (along with the PARK loci as described by Lansbury and Brice, 2002). The BLAST search output was filtered using thresholds of e<10-5 (FIG. 1) and e<10-3 (FIG. 2).

The following human genes were identified as a result of this screen: CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, and SMARCA1.

### Example 2

### Identification of Suppressors and Enhancers of Alpha Synuclein-Mediated Toxicity

Phenotype MicroArray™ (Biolog, Inc., Hayward, Calif.) was employed to evaluate the effects of various compounds in yeast expressing one or two copies of alpha synuclein.

In yeast expressing two copies of alpha synuclein (associated with severe growth defects), the following compounds were identified as suppressors of alpha synuclein-mediated toxicity: nordihydroguaiaretic acid (fungicide; lipoxygenase inhibitor), ibuprofen (prostaglandin synthetase inhibitor), D,L-a-hydroxy-butyric acid, m-cresol (membrane detergent), hexachlorophene (electron transporter), ruthenium red (mitochondrial Ca++ porter), sodium metasilicate (toxic anion), sodium metavanadate (toxic anion), sodium cyanide (toxic anion), and tetracycline (antibiotic). Compounds that alleviate alpha synuclein-mediated toxicity constitute potential therapeutics for the treatment of Parkinson's Disease.

In yeast expressing one copy of alpha synuclein (associated with moderate growth defects), the following compounds and conditions were identified as enhancers of alpha synuclein-mediated toxicity: modulators of osmotic sensitivity, detergents, oxidants, drugs affecting transport, carboxin, oxycarboxin, harmane, monensin, dodecyltrimethyl ammonium bromide, cetylpyridinium chloride, saponin, Phe-Met, L-Isoleucine, diamide, plumbagin, D-Sphingosine, chelerythrine, anisomycin, disulfuram, thiophosphate, dithiophosphate, sodium phosphate pH 7, sodium benzoate pH 5.2, and sodium thiosulfate.

### Example 3

### Identification of Suppressors of Huntingtin-Mediated Toxicity

Phenotype MicroArray™ (Biolog, Inc., Hayward, Calif.) was employed to evaluate the ability of various compounds to rescue viability in yeast expressing a toxic amount and form of huntingtin. The following compounds were identified as suppressors of huntingtin-mediated toxicity: clioquinols (8-Hydroxyquinoline, 5,7-Dichloro-8-hydroxy-quinaldine, and 8-Hydroxy-5-nitroquinoline), histidine-containing dipeptides (chelators), nordihydroguaiaretic acid (fungicide, lipoxygenase inhibitor), m-cresol (membrane detergent), and guanidine hydrochloride (chaotropic agent). Bioscreen C MBR (Labsystem Corp, Helsinki, Finland) was also used to evaluate the effects of the clioquinols. Similar to the results observed in the Phenotype MicroArray™ screen, the three clioquinols (8-Hydroxyquinoline, 5,7-Dichloro-8-hydroxy-quinaldine, and 8-Hydroxy-5-nitroquinoline) alleviated huntingtin-mediated toxicity in yeast. Compounds that alleviate huntingtin-mediated toxicity constitute potential therapeutics for the treatment of Huntington's Disease.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1.-15. (canceled)

16. A method of identifying a compound that inhibits aS mediated toxicity, the method comprising:
providing a yeast cell expressing an amount of aS that reduces viability of the cell;
contacting the cell with candidate agent selected from the group consisting of a fungicide, lipoxygenase inhibitor, prostaglandin synthetase inhibitor, membrane detergent, electron transporter, mitochondrial Ca++ porter, toxic anion, and antibiotic; and
determining whether the candidate agent enhances viability of the cell, to thereby identify a compound that inhibits aS mediated toxicity.

17.-18. (canceled)

19. A method of identifying a compound that inhibits aS mediated toxicity, the method comprising:
identifying a candidate agent that stimulates the expression or activity of a protein encoded by a gene selected from the group consisting of CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, and SMARCA1;
contacting a cell expressing aS with the candidate agent; and
determining whether the candidate agent enhances viability of the cell, to thereby identify a compound that inhibits aS mediated toxicity.

20. A method of identifying a compound that inhibits aS mediated toxicity, the method comprising:
providing a cell expressing aS and not expressing a wild type gene selected from the group consisting of CHD5, CPT2, CTH, AMPD2, AMPD1, CHD1L, NIT1, ACOX2, NIT2, ENPP6, SMARCA5, ENPEP, SMARCAD1, ACOX3, ARTS-1, LNPEP, LRAP, CHD1, SOD2, HBS1L, ENPP3, ENPP1, EEF1A1, ENPP5, CROT, UBE2H, RAD54B, CRAT, SMARCA2, CHAT, ERCC6, HELLS, SUPV3L1, BTAF1, AMPD3, CPT1A, EP400, TRHDE, CHD4, ATP7B, CHD2, ANPEP, KIAA1259, HAGH, GSPT1, SRCAP, FLJ12178, ACQX1, NPEPPS, PEMT, CPT1C, SMARCA4, EEF1A2, ARFRP1, CHD6, CPT1B, GSPT2, ATP7A, and SMARCA1, such that the cell has reduced viability as compared to a cell not expressing aS and expressing the wild type gene;
contacting the cell with a candidate agent; and
determining whether the candidate agent enhances viability of the cell, to thereby identify a compound that inhibits aS mediated toxicity.

21. (canceled)

22. The method of claim 19, wherein the wild type gene is selected from the group consisting of ACOX2, ACOX3, ATP7A, ATP7B, CPT1A, CPT1B, CPT1C, CPT2, CRAT, CROT, and SOD2.

23. The method of claim 19, wherein the wild type gene is ACOX2 or ACOX3.

24. The method of claim 19, wherein the wild type gene is ATP7A or ATP7B

25. The method of claim 19, wherein the wild type gene is CPT1A, CPT1B, CPT1C, CPT2, CRAT, or CROT.

26. The method of claim 19, wherein the wild type gene is SOD2.

27. The method of claim 20, wherein the wild type gene is selected from the group consisting of ACOX2, ACOX3, ATP7A, ATP7B, CPT1A, CPT1B, CPT1C, CPT2, CRAT, CROT, and SOD2.

28. The method of claim 20, wherein the wild type gene is ACOX2 or ACOX3.

29. The method of claim 20, wherein the wild type gene is ATP7A or ATP7B

30. The method of claim 20, wherein the wild type gene is CPT1A, CPT1B, CPT1C, CPT2, CRAT, or CROT.

31. The method of claim 20, wherein the wild type gene is SOD2.
